(19) United States
(12) Patent Application Publication
(10) Pub. No.: US 2009/0054372 A1
(43) Pub. Date: Feb. 26, 2009

(54) Combination therapy for weight loss

(76) Inventor: Ivan L. Goldsmith, Las Vegas, NV (US)

Correspondence Address: VENABLE LLP, P.O. BOX 34385, WASHINGTON, DC, 20043-9998, US

(21) Appl. No.: 12/076,720

(22) Filed: Mar. 21, 2008

Related U.S. Application Data
(60) Provisional application No. 60/896,125, filed on Mar. 21, 2007.

(51) Int. Cl. A61K 31/357; A61K 31/714; A61K 31/51; A61K 31/525; A61K 31/4415; A61P 3/10; A61P 3/04

(52) U.S. Cl. 514/52; 514/454; 514/276; 514/251; 514/355; 514/345

(57) ABSTRACT

The present invention is directed to methods and kits for achieving or maintaining weight loss. These kits and methods can include administering a pharmaceutical composition comprising phentermine, topiramate and a B vitamin to a subject in need thereof.

### BACKGROUND

A growing segment of the population, both in the U.S. and worldwide, suffers from obesity. Suggesting that the solution lies simply in diet and exercise trivializes the problem and fails to address many of the underlying causes of obesity.

Individuals struggling with weight problems often suffer from a variety of metabolic syndromes that make managing weight difficult. Metabolic malfunction may be a result of an inherent, internal physiological problem, such as hypothyroidism, or be induced by medications that an individual may take for treatment of a different health concern. Many neurological conditions are worsened by increased weight, including migraines, epilepsy, and spine conditions. Regardless of the source of the disease, there remains an unmet need for practical obesity treatments that are accessible, inexpensive, easy to administer, and easy for the patient to follow. This includes non-surgical options when diet and exercise are unsuccessful or insufficient.

### SUMMARY

Embodiments of the present invention are directed to methods and kits for achieving or maintaining weight loss in a subject comprising concurrently administering phentermine, topiramate and a B vitamin to a subject in need thereof. In some embodiments, the phentermine, topiramate and a B vitamin are in a single dosage form while in others they are in more than one dosage form. For example, the phentermine and topiramate can be in tablet form, either together or individually, and the B vitamin can be administered via injection.

The present invention, in some embodiments, is also directed to methods of determining the proper weight loss regimen for a subject in need thereof by evaluating the subject's health to determine if a daily weight loss regimen comprising phentermine, topiramate, and a B vitamin is safe and then administering the daily regimen to the subjects for whom the regimen has been determined to be safe.

### DETAILED DESCRIPTION

One embodiment of the present invention is directed to a method for weight loss that includes the administration of a multi-drug dosage form. The dosage form can include an appetite-suppressant, such as phentermine, in combination with an anticonvulsant such as topiramate, and a B vitamin, such as B12. These components can also be administered in separate dosage forms. For example, each component can be administered in an individual dosage form that does not contain the other components.

It has been found that the administration of these components is effective in inducing weight loss in subjects. Effective dosages include from about 5 to about 80 mg phentermine and about 5 to about 1000 mg topiramate. The term “about,” when used in conjunction with a dosage amount, means plus or minus ten percent of the specified amount. Example dosages of phentermine include, but are not limited to, about 5, 10, 15, 20, 25, 30, 35, 45, 50, or 55 mg per day. Example dosages of topiramate include, but are not limited to, about 50, 75, 100, 125, 150, 175, 200, 225, or 250 mg per day. A desirable dosage is about 37.5 mg or 40 mg phentermine or less and about 25 mg topiramate or more and about 1 mg of B12. According to one embodiment of the present invention, dosages do not exceed about 80 mg of phentermine and about 400 mg of topiramate.

While not wishing to be bound by a single theory, it is believed that the administration of phentermine, topiramate, and B12 in a single dosage form (hereinafter a “combination dosage form”) or concurrently using multiple dosage forms offers surprising advantages over other weight loss therapies that require the administration of multiple components at varying time intervals.

As used herein, “concurrently” or “concurrent” in relation to the administration of phentermine, topiramate, and B12 means that the components are administered within a specific time interval. For co-administration of separate dosage forms, the components can be administered within fifteen minutes or less of each other. Alternatively, the interval may be longer, for example, within one hour or less of each other, within six hours or less of each other, within twelve hours or less of each other, or within the same day. Concurrent administration refers to a time interval between administration of the active ingredients that is short enough to produce the desirable pharmacokinetic interactions described herein. For a single dosage form embodiment, the time interval is zero as all components are administered at the same time. In some embodiments, the concurrent administration is daily for a period of time sufficient to produce or maintain the desirable weight loss effects. For example, administration can be daily for at least about two, or three, or four or more months.

This single dosage form or concurrent administration can increase patient compliance since only one dosage form or treatment is necessary. If a single dosage form is used, there is also increased ease for a user, eliminating the need for managing multiple, individual medications.

It is further believed that the addition of B12 enhances the fat-burning effects of phentermine and topiramate. For example, B12 may help delay weight loss plateaus that can be experienced with the use of phentermine alone.

B12 is necessary for the regeneration of folic acid, aiding the function of multiple cellular enzymes and is critical for metabolism and neurological systems. In particular, B12 is involved in the synthesis of molecules necessary for the production of energy and may have putative mood modulating activities. Based on current literature B12 improves energy levels that may assist exercise and prevent hunger. It also has been shown to improve activity and energy in patients with chronic fatigue.

The dosage forms discussed herein include pills, tablets, capsules, liquids, and any other pharmaceutical composition in which the active ingredients of the present formulation are administered to a patient.

Many individuals suffering from obesity exhibit a vitamin B12 deficiency, which can contribute to food cravings and the inability to achieve feelings of satiety. Remedying this deficiency can help achieve desired weight loss. B12 deficiency lowers red blood cell count, which results in decreased energy, insomnia, palpitations. Administering B12 in conjunction with phentermine can ensure that energy levels remain high and lessen likelihood of possible heart palpitations. Because phentermine is known to raise metabolism, the resulting enhanced metabolic performance in a patient after administration of phentermine can in turn aid in the absorption of B12. Studies have suggested that B12 may also help in the management of sleep-wake rhythm disorders which any appetite suppressant like phentermine can cause or exacerbate.

The formulation of embodiments of the present invention has achieved surprising efficacy and significant commercial success. Over 500 patients using the combination dosage form have averaged 20 lbs weight loss over 12 weeks. Data from individual patients is presented below, in Table 1. The patients in table 1 were administered 40 mg phentermine, 25 mg topiramate, and 1 mg B12 in the form of a single dosage pill. The dosage pill was in the form of a capsule containing 140 mg, with contents as follows:
Cyanocobalamin (B-12) 1 mg,
Topiramate 25 mg,
Phentermine 40 mg, and
Methylcellulose (Filler) 74 mg.

TABLE 1
Combination Dosage Form

| Age | | | | | |
|---|---|---|---|---|---|
| (years)/ | Starting | Ending | Weight | Duration | % body |
| Gender | Weight (lb) | Weight (lb) | Loss (lb) | (days) | wt loss |
| 21/F | 197.5 | 168.7 | 28.8 | 99 | −20% |
| 35/F | 214.6 | 200.9 | 13.7 | 84 | −18% |
| 47/F | 202.1 | 192.6 | 9.5 | 28 | −19% |
| 42/M | 230 | 221 | 9 | 66 | −17% |
| 22/F | 146.4 | 132.6 | 13.8 | 75 | −27% |
| 29/F | 217 | 206.9 | 10.1 | 68 | −18% |
| 25/F | 172.6 | 150 | 22.6 | 104 | −23% |
| 42/F | 234.1 | 195.3 | 38.8 | 119 | −17% |
| 31/M | 240.5 | 233.3 | 7.2 | 63 | −16% |
| 56/F | 184 | 175.4 | 8.6 | 21 | −21% |
| 34/F | 149.9 | 142.3 | 7.6 | 36 | −26% |

As calculated from the table, patients lost an average of 20% of their body weight, or 15.43 pounds in 69 days. Patients were not given any specific exercise regimen, and were advised to follow a high-protein, low-carbohydrate diet, with no set calorie recommendations. As evidenced by Table 1, the therapy as set forth herein is surprisingly effective.

In some embodiments, 60% of patients with a body mass index greater than 30 taking a combination of phentermine/topiramate with modest exercise times per week and a low CHO (45 grams or less) can lose 11% or more of their total body weight after three months. These persons can also show improvements in their triglycerides, HDL, HgBAlc, fasting insulin, and waist circumference (e.g., a decline of at least three inches). These results can be similar to or surpass the results obtained using surgical options, such as Lap Band (see www.lapband.com; available from Allergan, Inc., Irvine, Calif.).

It has been observed that individuals taking the combination dosage form show improved weight loss compared to patients taking phentermine, topiramate, and B12 in separate dosage forms. It is hypothesized that in some embodiments the administration of B12 in one dosage form with phentermine and topiramate is more effective than B12 administered in a separate dosage form.

However, in some embodiments, the present invention is also directed to embodiments where B12 is administered in a separate dosage form, for example by injection, as part of a dosing regimen using the combination dosage forms described herein. In some embodiments, the B12 can be administered separate from the combination dosage form as a supplemental dose or instead of the B12 described herein as part of the combination dosage form.

It is further believed that the active ingredients are more easily released when administered in a single dosage form and more readily available to the patient because of simultaneous absorption and synchronized pharmacokinetics. As mentioned above, the increased metabolic rate induced by phentermine can aid in the absorption of B12 and may, in turn, reduce cravings.

In one embodiment of the invention, the dosage form can have about 40 mg phentermine, about 25 mg topiramate, and about 1 mg of B12 (as used in Table 1). In another embodiment of the invention, an additional dosage form (hereinafter “supplemental dosage form”) containing topiramate and B12 can be administered at a separate time than the combination dosage form. For instance, the combination dosage form can be taken in the morning, and the supplemental dosage form taken in the evening. In a further embodiment, two supplemental dosage forms can be administered at times where the patient exhibits increased susceptibility to binges.

For instance, a supplemental dosage form can be taken in the afternoon, and a second supplemental dosage form can be taken late evening. Food cravings are common at these times, in part due to circadian rhythm cycles and hormonal fluctuations. For instance, many individuals exhibit tiredness in the afternoon, and will crave and consume foods high in sugar and caffeine for an energy boost. It is also common for individuals battling obesity to confuse fatigue with hunger, triggering excess food consumption as energy levels dip in the afternoon. In addition, many overweight or obese individuals suffer from nighttime carbohydrate cravings and as a result, consume disproportionately large quantities of food later in the day. The supplemental dosage form can help reduce these cravings and assist in weight loss.

In accordance with embodiments of the invention, a supplemental dosage form can be administered when these cravings appear, regardless of what time of day cravings occur. The number of supplemental dosage forms taken by patients can vary, as long as the cumulative dosage of topiramate does not exceed safe maximum dosage. Safe maximum dosages for an average adult are indicated as about 80 mg phentermine and about 1000 mg topiramate, according to current medical evidence.

In some embodiments of the invention, the dosages are not administered with food. For example, the dose (combination or supplemental dosage form) can be administered approximately 30 minutes prior to a meal. It is believed that the release and absorption of active ingredients is greater when not coupled to the consumption of food.

In embodiments of the invention, the administration of the supplemental dosage form can help curb food cravings that may occur in the afternoon, evening, or both. The combination dosage form is recommended for morning use because phentermine is a stimulant and may keep users awake. An exemplary dosage for the supplemental dosage form can be about 25 mg topiramate and optionally about 1 mg B12. The dosage of topiramate can also be increased to a maximum of about 400 mg per day.

A segment of the population that suffers, perhaps disproportionately, from obesity is individuals who are nighttime workers (i.e. doctors, nurses, policemen, etc.) This is likely attributed to a flux in hormones which occurs because the schedule of a night worker is contrary to the natural circadian rhythm cycle. Altered hormone production tends to increase food cravings.

For night workers, the combination dosage form can be administered in the afternoon or evening hours, such as prior to a work shift. The phentermine in the combination dosage form may increase alertness, which is an added benefit, particularly when one is trying to be awake at time usually reserved for sleep. The supplemental dosage form, if taken, can be administered, for example, prior to or after consumption of the worker's third meal, or perhaps about 12 hours after the combination dosage form is taken. Ultimately, a night worker can take the combination dosage form at beginning of his wakeful period, and the supplemental dosage form, if part of the individual's regimen, taken at the later part of his or her working day.

In accordance with another embodiment of the invention, dosage forms can be administered one, two, three or more times daily. For instance, a dosage form can be administered approximately 10-60 minutes prior to each meal. This regimen can include the administration of a combination dosage form prior to breakfast, and a supplemental dosage form prior to lunch and dinner. By taking the dosage prior to a meal, the active ingredients have time to be released and absorbed by the body, and can help prevent overeating at meals.

A thrice daily regimen, in particular, can help break the connection between the sight of food and desire to ingest it. It may also help overcome the emotional feeling of the need to eat at a designated meal time. Once this is accomplished the patient can start focus on eating only when hungry, helping to establish healthy eating habits for long-term weight loss and maintenance.

In some patients, it may be desirable to administer the combination dosage twice daily. In one embodiment of the invention the combination dosage is administered before breakfast and before lunch. A supplemental dosage form can also be administered, for example, before dinner.

Before beginning or selecting a dosage regimen, it may be beneficial to first assess the patient to confirm that the phentermine dosage can be tolerated. Such tests can include a careful examination of the patient's cardiac health.

In a further embodiment of the invention, vitamins can be administered intravenously. The injection can include a complex of vitamins, such as B12 and other B vitamins. The dosage of B12 may be from about 5 micrograms to about 1 mg. Specific dosages of B12 can be about 0.25 mg, about 0.5 mg, or about 1.0 mg. In one embodiment of the invention, the composition of the injection is as follows (per 1 cc): Vitamin C-50 mg, B1-50 mg, B2-5 mg, B3-50 mg, B5-5 mg, B6-5 mg, B12-1000 mcg, Methionine-12.5 mg, Inositol-25 mg, Choline-25 mg, Lidocaine-10 mg.

As discussed above, many individuals suffering from metabolic syndromes, and those who take gastritis medication have impaired B12 absorption. Vitamin injections can help remedy the deficiency, and this may reduce cravings. It is also believed that the vitamin complex enhances the fat-burning effects of phentermine and topiramate. In one embodiment of the invention, the vitamin injection is administered once a week. Absorption of B12 is greater when administered via injection vs. orally, likely due to pharmacokinetics.

The treatment regimen can also include vitamins in dosage form. Vitamin dosage forms can be in lieu of, or in addition to the vitamin injection, depending on individual needs. The addition of B12 to the regimen can improve the overall health of the patient, in addition to enhancing weight loss. Increased overall wellness can cause a reduction in food cravings, as many cravings are triggered by emotional causes, such as stress and depression. Adding vitamins as part of the weight loss regimen described herein can help achieve and maintain weight loss by eliminating some the triggers of binge eating or overeating.

Hormone injections can also be administered as part of the weight-loss treatment described herein. As hormones are often a contributing factor in food cravings, it is believed that hormone injectables, such as human growth hormone and human chorionic gonadotropin (HCG) can aid in controlling cravings and in weight loss. In accordance with one embodiment of the invention, a hormone injection can be administered once per week.

The treatment set forth herein can be administered as follows. Upon arrival to a clinic, a patient is assessed for treatment. The assessment includes determination of vital signs, BMI, abdominal circumference, and history of diet/exercise methods which have failed. The first dose can be administered in the clinic and a set of vital signs checked at 30 minutes. After this the drug is supplied, such as at an on-site pharmacy, and the patient returns home to take further doses as scheduled. Follow-up visits are at two week intervals for the first three months. At that time the program is complete, but patients are free to continue maintenance therapy. The regimen is safe for up to two years and beyond.

The compounds of the invention can be formulated as pharmaceutical compositions and administered to a subject in need of treatment, for example a mammal, such as a human patient, in a variety of forms adapted to the chosen route of administration, for example, orally, nasally, intraperitoneally, or parenterally, by intravenous, intramuscular, topical or subcutaneous routes, or by injection into tissue.

Thus, compounds of the invention may be systemically administered, e.g., orally, in combination with a pharmaceutically acceptable vehicle such as an inert diluent or an assimilable edible carrier, or by inhalation or insufflation. They may be enclosed in hard or soft shell gelatin capsules, may be compressed into tablets, or may be incorporated directly with the food of the patient's diet. For oral therapeutic administration, the compounds may be combined with one or more excipients and used in the form of ingestible tablets, buccal tablets, troches, capsules, elixirs, suspensions, syrups, wafers, and the like. The compounds may be combined with a fine inert powdered carrier and inhaled by the subject or insufflated. Such compositions and preparations may contain at least 0.1% of a compound or compounds of the invention. The percentage of the compositions and preparations may, of course, be varied and may conveniently be between about 2% to about 60% of the weight of a given unit dosage form. The amount of compounds in such therapeutically useful compositions is such that an effective dosage level will be obtained.

In order to facilitate administration of the therapy, the dosage forms can be provided in a kit with instructions for use. Instructions include any form of labeling, indications for use, and regulations, and the instructions can be printed on the kit or in labels accompanying the kit. The kit can include the combination dosage form alone, the combination dosage form and supplemental dosage form, either dosage form with vitamins, both dosage forms with vitamins, or other desirable combinations. The kit can also include vitamins, in an oral and/or injectable form. The kit can provide, for example, a 30-day supply of the combination dosage form, supplemental dosage form, and vitamins. This allows easy self-administration of oral dosage forms, and quick administration of the injection at a location chosen by the patient (e.g. a doctor's office). This gives the patient the freedom of choosing any health care professional to administer the injection, rather than having to go to a weight loss clinic, which is particularly convenient when the patient is not in close proximity to a clinic.

The weight loss therapy described herein, in any of its embodiments, can be used to treat a patient suffering from one or more of obesity, a metabolic syndrome, and binge disorder. A metabolic syndrome can be considered as any deviation from normal metabolic function. A binge disorder is typified by the consumption of large quantities of food in a small space of time, often beyond the point of fullness. Obese people binge but typically do not purge, as they have a psychological dependence on the act of ingestion and revulsion of the act of purging.

The therapy described herein can also be used to treat any form of disordered eating (e.g. an eating disorder), regardless of the underlying causes. Disordered eating includes the consumption of significantly more calories than the body requires to function.

Embodiments of the present invention are also directed to methods of determining, predicting, or otherwise finding the proper weight loss regimen for a subject in need thereof. A subject, for example a patient in a weight loss clinic, can be tested to determine if their cardiac health and overall health is sufficient to tolerate a weight loss regimen using phentermine, topiramate, and/or a B vitamin without undue risk. If this subject has an acceptable level of risk, then the patient is administered a weight loss regimen as disclosed herein. Should the patient have an unacceptable level of risk, as determined by a medical professional, then the patient will can be administered a more appropriate weight loss therapy.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that the invention may be practiced otherwise than as specifically described. All articles, websites, or patents cited herein are incorporated by reference in their entirety.

We claim:

1. A method of achieving or maintaining weight loss in a subject comprising concurrently administering phentermine, topiramate and a B vitamin to a subject in need thereof.

2. The method of claim 1, wherein the dosage of phentermine is about 37.5 mg.

3. The method of claim 1, wherein the dosage of topiramate is about 25 mg.

4. The method of claim 1, wherein the B vitamin is B12 in a dosage of about 1 mg.

5. The method of claim 1, wherein the phentermine, topiramate and a B vitamin are administered in a single pharmaceutical dosage form consisting essentially of phentermine, topiramate and a B vitamin.

6. The method of claim 1, wherein the phentermine, topiramate and a B vitamin are administered to a subject daily until sufficient weight loss has been achieved.

7. The method of claim 1, wherein the phentermine, topiramate and a B vitamin are administered in the morning.

8. The method of claim 7, further comprising administering an additional dose of topiramate and vitamin B12 in the evening.

9. The method of claim 1, further comprising administering a weekly vitamin injection.

10. The method of claim 9, wherein the vitamin injection comprises 50 mg of Vitamin C, 50 mg of Vitamin B1, 5 mg of Vitamin B2, 50 mg of Vitamin B3, 5 mg of Vitamin B5, 5 mg of Vitamin B6, 1000 micrograms of Vitamin B12, 12.5 mg of Methionine, 25 mg of Inositol, 25 mg of Choline, and 10 mg of Lidocaine.

11. The method of claim 1, wherein the phentermine, topiramate, and B12 are in a single dosage form.

12. The method of claim 1, further comprising a hormone treatment.

13. The method of claim 1, wherein the subject is suffering from obesity, a metabolic syndrome, or a binge eating disorder.

14. A weight loss kit comprising a container and phentermine, topiramate, and a B vitamin, wherein the phentermine, topiramate, and a B vitamin are in one or more pharmaceutical compositions.

15. The kit of claim 14, wherein the phentermine, topiramate, and B12 are in a single dosage form.

16. The kit of claim 14, wherein the dosage of phentermine is about 40 mg.

17. The method of claim 14, wherein the dosage of topiramate is about 25 mg.

18. The kit of claim 14, wherein the B vitamin is B12 in a dosage of about 1 mg.

19. The kit of claim 14, further comprising a second pharmaceutical composition comprising about 25 mg topiramate and about 1 mg of B12.

20. A method of determining the proper weight loss regimen for a subject in need thereof comprising evaluating the subject's health to determine if a daily weight loss regimen comprising phentermine, topiramate, and a B vitamin is safe and then administering the daily regimen to the subjects for whom the regimen has been determined to be safe.
